(19) United States
(12) Patent Application Publication
(10) Pub. No.: US 2026/0285860 A1
(43) Pub. Date: Sep. 24, 2026

(54) INHIBITORS OF cGAS AND USES THEREOF

(71) Applicant: The Trustees of Columbia University in the City of New York, New York, NY (US)

(72) Inventors: Lindsay CAPRIO, New York, NY (US); Benjamin IZAR, New York, NY (US); Donald LANDRY, New York, NY (US); Shi-Xian DENG, New York, NY (US)

(21) Appl. No.: 19/676,059

(22) Filed: May 13, 2026

Related U.S. Application Data
(63) Continuation-in-part of application No. PCT/US2024/056642, filed on Nov. 20, 2024.
(60) Provisional application No. 63/601,333, filed on Nov. 21, 2023.

(51) Int. Cl. C07D 471/04; A61K 31/444; A61P 35/00; A61P 35/04

(52) U.S. Cl. CPC C07D 471/04; A61K 31/444; A61P 35/00; A61P 35/04

(57) ABSTRACT

Compounds for inhibiting cGAS, including those having the following structure and methods of use thereof in treating tumors:

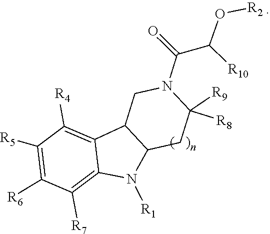

### CROSS-REFERENCE TO RELATED APPLICATIONS

This application is a continuation in part of PCT International Application No. PCT/US2024/056642, filed Nov. 20, 2024, and claims benefit of U.S. Provisional Application No. 63/601,333, filed Nov. 21, 2023, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

The entire content of U.S. Publication No. US 2021/01155625 and US 2022/0185812 are hereby incorporated by reference.

### BACKGROUND OF THE INVENTION

Tumors characterized by high levels of chromosomal instability (CIN) are associated with poorer survival across diverse cancer types and are resistant to an array of cancer treatment modalities, particularly novel immunotherapies, however mechanisms of resistance remain to be elucidated.

A well-established consequence of CIN is the formation of micronuclei (MN) which harbor excessive dsDNA. These MN are highly rupture-prone, releasing dsDNA to the cytosol where it is sensed and activates cGAS, which converts dsDNA to cGAMP. cGAMP binds to STING which subsequently triggers a cascade resulting in production of type I interferons (IFNs) that are strong activators of adaptive anti-viral and anti-tumor immunity.

This represents a long-standing biological conundrum: if CIN^{high} tumors activate the cGAS-STING pathway, one would expect improved anti-tumor immunity, yet the opposite is true, and is exemplified by inherent resistance to ICB in CIN^{high} tumor types such as LKB1-deficient non-small cell lung cancer (NSCLC). Mechanistically, it has been previously shown that chromosomally unstable tumors re-wire downstream STING signaling to i) enact pro-metastatic signaling through activation of non-canonical NF-kB activation and ii) form an immunosuppressive microenvironment through the degradation of exported cGAMP to adenosine in the tumor microenvironment.

### BRIEF SUMMARY OF THE INVENTION

The present invention provides compound having the following structure:

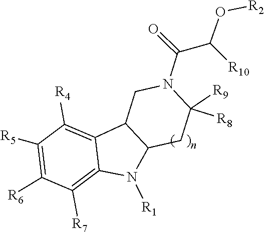

wherein
n is 1 or 2;
R₁ is hydrogen, (C₁-C₆)alkyl, (C₂-C₆)alkenyl, (C₂-C₆)alkynyl, CH₂CH₂OR₃, or fluoro (C₁-C₆)alkyl;
R₃ is hydrogen, halogen, (C₁-C₆)alkyl, (C₂-C₆)alkenyl, or (C₂-C₆)alkynyl;
R₅ is hydrogen, halogen, (C₁-C₆)alkyl, (C₂-C₆)alkenyl, (C₂-C₆)alkynyl, (C₁-C₆)alkoxy, fluoro(C₁-C₆)alkyl, fluoro(C₁-C₆)alkoxy, or cyano;
R₆ is hydrogen, halogen, cyano, (C₁-C₆)alkyl, (C₂-C₆)alkenyl, (C₂-C₆)alkynyl, or fluoro (C₁-C₆)alkyl;
R₇ is hydrogen, halogen, (C₁-C₆)alkoxy, fluoro(C₁-C₆)alkoxy, cyano, (C₁-C₆)alkyl, (C₂-C₆)alkenyl, (C₂-C₆)alkynyl, or fluoro(C₁-C₆)alkyl;
R₈ and R₉ are hydrogen, halogen, or (C₁-C₆)alkyl;
R₁₀ is hydrogen, halogen, (C₁-C₆)alkyl, (C₂-C₆)alkenyl, or (C₂-C₆)alkynyl;
   wherein when n is 1:
   R₂ is H, or a substituted or unsubstituted amino acid ester or α-hydroxy ester;
   R₄ is a cyclic group substituted with one or more amines, or substituted with amino acids via the α-N
   wherein
      (i) when R₂ is H, R₄ is a cyclic group substituted with one or more amino acids via the α-N; and
      (ii) when R₂ is a substituted or unsubstituted amino acid ester or α-hydroxy ester, R₄ is a cyclic group substituted with one or more amines, or substituted with amino acids via the α-N;
   wherein when n is 2:
   R₂ is a substituted or unsubstituted amino acid ester or α-hydroxy ester, hydrogen, (C₁-C₆)alkyl, (C₂-C₆)alkenyl, (C₂-C₆)alkynyl, or CH₂CH₂OR₁₁, or a 4- to 6-membered aliphatic ring containing an O;
   R₁₁ is hydrogen, (C₁-C₆)alkyl, (C₂-C₆)alkenyl, or (C₂-C₆)alkynyl;
   R₄ is hydrogen, halogen, (C₁-C₆)alkoxy, cyano, amino, an optionally substituted (C₁-C₆)alkyl, optionally substituted mono- or bicyclic heterocycle, optionally substituted phenyl, optionally substituted benzyl, (C₁-C₆) acylamino, or (C₁-C₆)N-alkyl carboxamido, wherein
      the optionally substituted alkyl, mono- or bicylclic heterocyle, phenyl, or benzyl groups are optionally substituted with amino, (C₁-C₆)mono- or dialkylamino, (C₁-C₆)alkoxy, or heterocyclic groups
or
a pharmaceutically acceptable salt or a pharmaceutically acceptable salt hydrate or deuterated analog thereof.

The present invention provides a compound having the following structure:

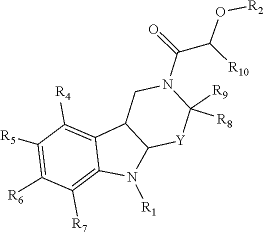

wherein
Y is —CH₂— or —CH₂—CH₂—; preferably, Y is —CH₂—;
R₁ is hydrogen, halogen, (C₁-C₆)alkyl, (C₂-C₆)alkenyl, (C₂-C₆)alkynyl, CH₂CH₂OR₃, or fluoro (C₁-C₆)alkyl;
   wherein when Y is —CH₂—; R₂ is a substituted or unsubstituted amino acid;
   wherein when Y is —CH₂—CH₂—; R₂ is a substituted or unsubstituted amino acid, hydrogen, (C₁-C₆)alkyl, (C₂-C₆)alkenyl, (C₂-C₆)alkynyl, or CH₂CH₂OR₃, or, taken together along with the atoms to which they are attached, R₂ and R₃ may form a 4- to 6-membered aliphatic ring;
R₃ is hydrogen, halogen, (C₁-C₆)alkyl; (C₂-C₆)alkenyl, or (C₂-C₆)alkynyl;
R₄ is hydrogen, halogen, (C₁-C₆)alkoxy, optionally substituted monocyclic heterocyclyl, cyano, optionally substituted phenyl, optionally substituted bicyclic heterocyclyl, amino(C₁-C₆)alkyl, (C₁-C₆)alkylamino(C₁-C₆)alkyl, (C₁-C₆)dialkylamino (C₁-C₆)alkyl, (C₁-C₆)alkoxy(C₁-C₆)alkyl, (C₁-C₆)hydrocarbyl, heterocyclyl(C₁-C₆)alkyl, benzyl, heterocyclyl-substituted benzyl, (C₁-C₆) alkylaminocarbonyl, or (C₁-C₆)acylamino;
R₅ is hydrogen, halogen, (C₁-C₆)alkyl, (C₂-C₆)alkenyl, (C₂-C₆)alkynyl; (C₁-C₆)alkoxy, fluoro(C₁-C₆)alkyl, fluoro(C₁-C₆)alkoxy, or cyano;
R₆ is hydrogen, halogen, cyano, (C₁-C₆)alkyl, (C₂-C₆)alkenyl, (C₂-C₆)alkynyl; or fluoro (C₁-C₆)alkyl;
R₇ is hydrogen, halogen, (C₁-C₆)alkoxy, fluoro(C₁-C₆)alkoxy, cyano, (C₁-C₆)alkyl, (C₂-C₆)alkenyl, (C₂-C₆)alkynyl; or fluoro(C₁-C₆)alkyl;
R₈ is hydrogen, halogen, or (C₁-C₆)alkyl, or, taken together with the carbon to which they are attached, R₈ and R₉ may form a 3- to 5-membered aliphatic carbocyclic ring;
R₉ is hydrogen, halogen, or (C₁-C₆)alkyl, or, taken together with the carbon to which they are attached, R₈ and R₉ may form a 3- to 5-membered aliphatic carbocyclic ring;
R₁₀ is hydrogen, halogen, (C₁-C₆)alkyl, (C₂-C₆)alkenyl, or (C₂-C₆)alkynyl, or, taken together along with the atoms to which they are attached,
wherein
(a) when R₂ is (C₁-C₆)alkyl, not all of R₄, R₅, R₆, and R₇ can be hydrogen;
(b) when R₁ is hydrogen, R₂ is methyl and R₅ is halogen, R₇ cannot be H or chloro; and
(c) when R₂ is (C₁-C₆)alkyl, and R⁵ is methoxy or carboxy, R⁷ cannot be H;
or a pharmaceutically acceptable salt of the compound.

A prodrug or a pharmaceutically acceptable salt having the following:

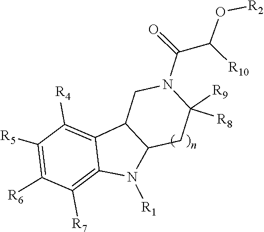

wherein
n is 1 or 2, preferably n is 1;
R₁ is hydrogen, halogen, (C₁-C₆)alkyl, (C₂-C₆)alkenyl, (C₂-C₆)alkynyl, CH₂CH₂OR₃, or fluoro (C₁-C₆)alkyl;
   wherein when n is 1, R₂ is a substituted or unsubstituted amino acid ester or α-hydroxy ester;
   wherein when n is 2, R₂ is a substituted or unsubstituted amino acid ester or α-hydroxy ester, hydrogen, (C₁-C₆)alkyl, (C₂-C₆)alkenyl, (C₂-C₆)alkynyl, or CH₂CH₂OR₃, or, a 4- to 6-membered aliphatic ring containing an O;
R₃ is hydrogen, (C₁-C₆)alkyl, (C₂-C₆)alkenyl, or (C₂-C₆)alkynyl;
R₄ is hydrogen, halogen, (C₁-C₆)alkoxy, cyano, amino, an optionally substituted mono- or bicyclic heterocycle, optionally substituted (C₁-C₆)alkyl, optionally substituted phenyl, optionally substituted benzyl, (C₁-C₆) acylamino, or (C₁-C₆)N-alkylcarboxamido,
wherein
   the alkyl, mono- or bicyclic heterocycle, phenyl, or benzyl groups are optionally substituted with amino, (C₁-C₆)mono- or dialkylamino, (C₁-C₆)alkoxy, heterocyclic groups
R₅ is hydrogen, halogen, (C₁-C₆)alkyl, (C₂-C₆)alkenyl, (C₂-C₆)alkynyl, (C₁-C₆)alkoxy, fluoro(C₁-C₆)alkyl, fluoro(C₁-C₆)alkoxy, or cyano;
R₆ is hydrogen, halogen, cyano, (C₁-C₆)alkyl, (C₂-C₆)alkenyl, (C₂-C₆)alkynyl, or fluoro (C₁-C₆)alkyl;
R₇ is hydrogen, halogen, (C₁-C₆)alkoxy, fluoro(C₁-C₆)alkoxy, cyano, (C₁-C₆)alkyl, (C₂-C₆)alkenyl, (C₂-C₆)alkynyl, or fluoro(C₁-C₆)alkyl;
R₈ and R₉ are hydrogen, halogen, or (C₁-C₆)alkyl, or, taken together with the carbon to which they are attached, R₈ and R₉ may form a 3- to 5-membered aliphatic carbocycle;
R₁₀ is hydrogen, halogen, (C₁-C₆)alkyl, (C₂-C₆)alkenyl, or (C₂-C₆)alkynyl,
or a pharmaceutically acceptable salt of the compound.

### BRIEF DESCRIPTION OF THE DRAWINGS

FIG. 1A is a general structure of the compound disclosed in the present invention.

FIG. 1B is a specific structure of the compound disclosed in the present invention in salt form.

FIG. 1C is compound G150.

### DETAILED DESCRIPTION OF THE INVENTION

Through genetically-mediated knockout of cGAS in CIN^{high} models, present invention restored sensitivity to ICB and restoration of pro-inflammatory signaling downstream of STING activation in chromosomally unstable tumor models, thus restoring protypical STING activation outputs even in CIN^{high} models.

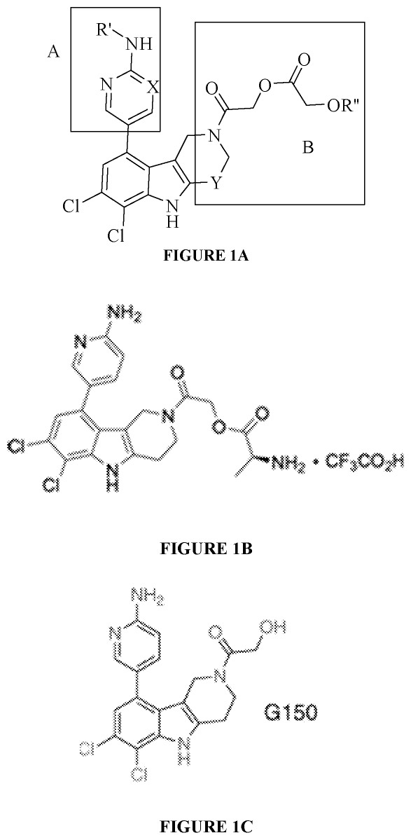

Thus, the present invention(s) are a set of chemically modified small-molecule inhibitors of cGAS. These inhibitors will improve aqueous solubility, cell permeability, and/or oral bioavailability through the addition of polar groups at R′ and R″ position (See FIG. 1A). A prodrug coupling with alanine at the amino group at the 2-position of the pyridine ring permits the amide bond of the prodrug to be hydrolyzed by an aminopeptidase while having improved solubility.

The present invention provides compound having the following structure:

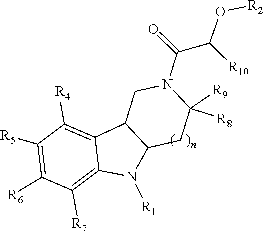

wherein
n is 1 or 2;
R₁ is hydrogen, (C₁-C₆)alkyl, (C₂-C₆)alkenyl, (C₂-C₆)alkynyl, CH₂CH₂OR₃, or fluoro (C₁-C₆)alkyl;
R₃ is hydrogen, halogen, (C₁-C₆)alkyl, (C₂-C₆)alkenyl, or (C₂-C₆)alkynyl;
R₅ is hydrogen, halogen, (C₁-C₆)alkyl, (C₂-C₆)alkenyl, (C₂-C₆)alkynyl, (C₁-C₆)alkoxy, fluoro(C₁-C₆)alkyl, fluoro(C₁-C₆)alkoxy, or cyano;
R₆ is hydrogen, halogen, cyano, (C₁-C₆)alkyl, (C₂-C₆)alkenyl, (C₂-C₆)alkynyl, or fluoro (C₁-C₆)alkyl;
R₇ is hydrogen, halogen, (C₁-C₆)alkoxy, fluoro(C₁-C₆)alkoxy, cyano, (C₁-C₆)alkyl, (C₂-C₆)alkenyl, (C₂-C₆)alkynyl, or fluoro(C₁-C₆)alkyl;
R₈ and R₉ are hydrogen, halogen, or (C₁-C₆)alkyl;
R₁₀ is hydrogen, halogen, (C₁-C₆)alkyl, (C₂-C₆)alkenyl, or (C₂-C₆)alkynyl;
   wherein when n is 1:
   R₂ is H, or a substituted or unsubstituted amino acid ester or α-hydroxy ester;
   R₄ is a cyclic group substituted with one or more amines, or substituted with amino acids via the α-N
   wherein
      (i) when R₂ is H, R₄ is a cyclic group substituted with one or more amino acids via the α-N; and
      (ii) when R₂ is a substituted or unsubstituted amino acid ester or α-hydroxy ester, R₄ is a cyclic group substituted with one or more amines, or substituted with amino acids via the α-N;
   wherein when n is 2:
   R₂ is a substituted or unsubstituted amino acid ester or α-hydroxy ester, hydrogen, (C₁-C₆)alkyl, (C₂-C₆)alkenyl, (C₂-C₆)alkynyl, or CH₂CH₂OR₁₁, or a 4- to 6-membered aliphatic ring containing an O;
   R₁₁ is hydrogen, (C₁-C₆)alkyl, (C₂-C₆)alkenyl, or (C₂-C₆)alkynyl;
   R₄ is hydrogen, halogen, (C₁-C₆)alkoxy, cyano, amino, an optionally substituted (C₁-C₆)alkyl, optionally substituted mono- or bicyclic heterocycle, optionally substituted phenyl, optionally substituted benzyl, (C₁-C₆) acylamino, or (C₁-C₆)N-alkyl carboxamido, wherein
   the optionally substituted alkyl, mono- or bicylclic heterocyle, phenyl, or benzyl groups are optionally substituted with amino, (C₁-C₆)mono- or dialkylamino, (C₁-C₆)alkoxy, or heterocyclic groups
or
a pharmaceutically acceptable salt or a pharmaceutically acceptable salt hydrate or deuterated analog thereof.

The present invention provides a compound having the following structure:

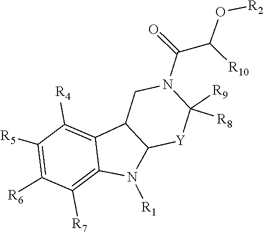

wherein
Y is —CH₂— or —CH₂—CH₂—; preferably, Y is —CH₂—;
R₁ is hydrogen, halogen, (C₁-C₆)alkyl, (C₂-C₆)alkenyl, (C₂-C₆)alkynyl, CH₂CH₂OR₃, or fluoro (C₁-C₆)alkyl;
   wherein when Y is —CH₂—; R₂ is a substituted or unsubstituted amino acid;
   wherein when Y is —CH₂—CH₂—; R₂ is a substituted or unsubstituted amino acid, hydrogen, (C₁-C₆)alkyl, (C₂-C₆)alkenyl, (C₂-C₆)alkynyl, or CH₂CH₂OR₃, or, taken together along with the atoms to which they are attached, R₂ and R₃ may form a 4- to 6-membered aliphatic ring;
R₃ is hydrogen, halogen, (C₁-C₆)alkyl; (C₂-C₆)alkenyl, or (C₂-C₆)alkynyl;
R₄ is hydrogen, halogen, (C₁-C₆)alkoxy, optionally substituted monocyclic heterocyclyl, cyano, optionally substituted phenyl, optionally substituted bicyclic heterocyclyl, amino(C₁-C₆)alkyl, (C₁-C₆)alkylamino(C₁-C₆)alkyl, (C₁-C₆)dialkylamino (C₁-C₆)alkyl, (C₁-C₆)alkoxy(C₁-C₆)alkyl, (C₁-C₆)hydrocarbyl, heterocyclyl(C₁-C₆)alkyl, benzyl, heterocyclyl-substituted benzyl, (C₁-C₆) alkylaminocarbonyl, or (C₁-C₆)acylamino;
R₅ is hydrogen, halogen, (C₁-C₆)alkyl, (C₂-C₆)alkenyl, (C₂-C₆)alkynyl; (C₁-C₆)alkoxy, fluoro(C₁-C₆)alkyl, fluoro(C₁-C₆)alkoxy, or cyano;
R₆ is hydrogen, halogen, cyano, (C₁-C₆)alkyl, (C₂-C₆)alkenyl, (C₂-C₆)alkynyl; or fluoro (C₁-C₆)alkyl;
R₇ is hydrogen, halogen, (C₁-C₆)alkoxy, fluoro(C₁-C₆)alkoxy, cyano, (C₁-C₆)alkyl, (C₂-C₆)alkenyl, (C₂-C₆)alkynyl; or fluoro(C₁-C₆)alkyl;
R₈ is hydrogen, halogen, or (C₁-C₆)alkyl, or, taken together with the carbon to which they are attached, R₈ and R₉ may form a 3- to 5-membered aliphatic carbocyclic ring;
R₉ is hydrogen, halogen, or (C₁-C₆)alkyl, or, taken together with the carbon to which they are attached, R₈ and R₉ may form a 3- to 5-membered aliphatic carbocyclic ring;
R₁₀ is hydrogen, halogen, (C₁-C₆)alkyl, (C₂-C₆)alkenyl, or (C₂-C₆)alkynyl, or, taken together along with the atoms to which they are attached,
wherein
(a) when R₂ is (C₁-C₆)alkyl, not all of R₄, R₅, R₆, and R₇ can be hydrogen;
(b) when R₁ is hydrogen, R₂ is methyl and R₅ is halogen, R₇ cannot be H or chloro; and
(c) when R₂ is (C₁-C₆)alkyl, and R₅ is methoxy or carboxy, R⁷ cannot be H;
or a pharmaceutically acceptable salt of the compound.

In some embodiments, R₁ is hydrogen or (C₁-C₆)alkyl; preferably R₁ is hydrogen.

In some embodiments, R₂ is a substituted amino acid.

In some embodiments, the substituted amino acid is substituted with amino, (C₁-C₆)alkylamino, meta-(C₁-C₆)dialkylamino, (C₁-C₆) alkoxy, hydroxy, halogen, ortho-cyano, meta-cyano, aminocarbonyl, methylenedioxy, ethylenedioxy, (C₁-C₆)acylamino, fluoro(C₁-C₆)acylamino, and hydroxy (C₁-C₆)alkylaminosulfonyl.

In some embodiments, R₂ is unsubstituted amino acid.

In some embodiments, R₂ is alanine.

In some embodiments, R₂ is

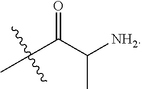 [SMILES: *C(=O)C(C)N]

In some embodiments, R₃ is hydrogen, halogen, or (C₁-C₆)alkyl.

In some embodiments, R₃ is hydrogen.

In some embodiments, R₄ is optionally substituted monocyclic heterocyclyl, optionally substituted phenyl or optionally substituted bicyclic heterocyclyl.

In some embodiments, wherein when n is 1, R₂ is an unsubstituted or α-substituted amino acid ester or α-hydroxy ester, preferably the amino acid or α-hydroxy ester is substituted with amino, (C₁-C₆)alkylamino, (C₁-C₆)alkoxy, hydroxy, halogen, carboxyamido, methylenedioxy, ethylenedioxy, (C₁-C₆)acylamino, fluoro(C₁-C₆)acylamino, and hydroxy (C₁-C₆)alkylaminosulfonyl.

In some embodiments wherein when n is 1, R₂ is an amino acid ester or α-hydroxy ester optionally unsubstituted or substituted on the α-heteroatom with an amino acid residue, α-hydroxy ester, α-(C₁-C₆)alkoxy ester (C₁-C₆)alkyl, (C₂-C₆)alkenyl, (C₂-C₆)alkynyl, amino(C₁-C₆)alkyl, hydroxy(C₁-C₆)alkyl, halo(C₁-C₆)alkyl, phenyl, benzyl, preferably R₂ is alanine.

In some embodiments, said optionally substituted monocyclic heterocyclyl may be substituted with one or more substituents chosen from: an amino acid, (C₁-C₆)alkyl, amino, cyano, (C₁-C₆)alkylamino, (C₁-C₆)alkoxy, oxo, fluoro (C₁-C₆)alkyl, halogen, hydroxy, and hydroxy(C₁-C₆)alkyl.

In some embodiments, said optionally substituted phenyl may be substituted with one or more substituents chosen from: an amino acid, amino, (C₁-C₆)alkylamino, meta-(C₁-C₆)dialkylamino, (C₁-C₆) alkoxy, hydroxy, halogen, ortho-cyano, meta-cyano, aminocarbonyl, methylenedioxy, ethylenedioxy, (C₁-C₆)acylamino, fluoro(C₁-C₆)acylamino, and hydroxy (C₁-C₆)alkylaminosulfonyl.

In some embodiments, said optionally substituted bicyclic heterocyclyl may be substituted with one or more substituents chosen from: an amino acid, (C₁-C₆)alkyl, hydroxy, and oxo; R5 is chosen from hydrogen, halogen, (C₁-C₆)alkyl, (C₁-C₆)alkoxy, fluoro(C₁-C₆)alkyl, fluoro(C₁-C₆)alkoxy, cyano.

In some embodiments, R₄ is substituted monocyclic heterocyclyl substituted with an amino acid, (C₁-C₃)alkyl, or amino group.

In some embodiments, the said optionally substituted monocyclic heterocyclyl is substituted with an amino acid or amino group.

In some embodiments, the amino acid is alanine.

In some embodiments, R₄ has the following structure:

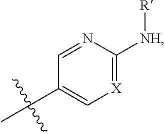

wherein X is C or N, wherein R′ is an amino acid.

In some embodiments, the amino acid is alanine.

In some embodiments, X is C.

In some embodiments, R₄ is

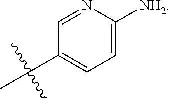 [SMILES: *c1ccc(N)nc1]

In some embodiments, R₅ is hydrogen, halogen, (C₁-C₆)alkyl, (C₂-C₆)alkenyl, (C₂-C₆)alkynyl.

In some embodiments, R₅ is hydrogen, halogen, or (C₁-C₆)alkyl.

In some embodiments, R₅ is hydrogen.

In some embodiments, R₆ is hydrogen, halogen, cyano, or (C₁-C₆)alkyl.

In some embodiments, R₆ is hydrogen, halogen, or (C₁-C₆)alkyl.

In some embodiments, R₆ is halogen, methyl, or trifluoromethyl.

In some embodiments, R₆ is halogen.

In some embodiments, R₆ is Cl.

In some embodiments, R₇ is hydrogen, halogen, cyano, or (C₁-C₆)alkyl.

In some embodiments, R₇ is hydrogen, halogen, or (C₁-C₆)alkyl.

In some embodiments, R₇ is halogen.

In some embodiments, R₇ is Cl.

In some embodiments, R₈ is hydrogen or (C₁-C₆)alkyl.

In some embodiments, R₈ is hydrogen.

In some embodiments, R₉ is hydrogen or (C₁-C₆)alkyl.

In some embodiments, R₉ is hydrogen.

In some embodiments, R₁₀ is hydrogen or halogen.

In some embodiments, R₁₀ is hydrogen.

The present invention provides a compound having the following structure:

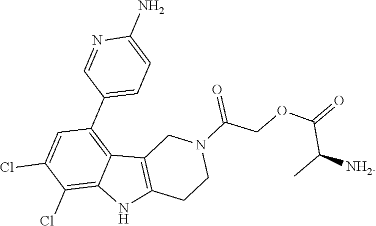 [SMILES: C[C@H](N)C(=O)OCC(=O)N1CCc2[nH]c3c(Cl)c(Cl)cc(-c4ccc(N)nc4)c3c2C1]

The present invention provides a prodrug or a pharmaceutically acceptable salt having the following:

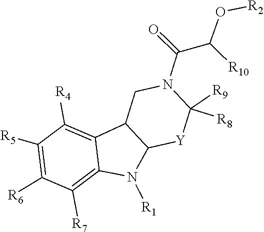

wherein
Y is —CH₂— or —CH₂—CH₂—; preferably, Y is —CH₂—;
R₁ is hydrogen, halogen, (C₁-C₆)alkyl, (C₂-C₆)alkenyl, (C₂-C₆)alkynyl, CH₂CH₂OR₃, or fluoro (C₁-C₆)alkyl;
   wherein when Y is —CH₂—; R₂ is a substituted or unsubstituted amino acid;
   wherein when Y is —CH₂—CH₂—; R₂ is a substituted or unsubstituted amino acid, hydrogen, (C₁-C₆)alkyl, (C₂-C₆)alkenyl, (C₂-C₆)alkynyl, or CH₂CH₂OR₃, or, taken together along with the atoms to which they are attached, R₂ and R₃ may form a 4- to 6-membered aliphatic ring;
R₃ is hydrogen, halogen, (C₁-C₆)alkyl; (C₂-C₆)alkenyl, or (C₂-C₆)alkynyl;
R₄ is hydrogen, halogen, (C₁-C₆)alkoxy, optionally substituted monocyclic heterocyclyl, cyano, optionally substituted phenyl, optionally substituted bicyclic heterocyclyl, amino(C₁-C₆)alkyl, (C₁-C₆)alkylamino(C₁-C₆)alkyl, (C₁-C₆)dialkylamino (C₁-C₆)alkyl, (C₁-C₆)alkoxy(C₁-C₆)alkyl, (C₁-C₆)hydrocarbyl, heterocyclyl(C₁-C₆)alkyl, benzyl, heterocyclyl-substituted benzyl, (C₁-C₆) alkylaminocarbonyl, or (C₁-C₆)acylamino;
R₅ is hydrogen, halogen, (C₁-C₆)alkyl, (C₂-C₆)alkenyl, (C₂-C₆)alkynyl; (C₁-C₆)alkoxy, fluoro(C₁-C₆)alkyl, fluoro(C₁-C₆)alkoxy, or cyano;
R₆ is hydrogen, halogen, cyano, (C₁-C₆)alkyl, (C₂-C₆)alkenyl, (C₂-C₆)alkynyl; or fluoro (C₁-C₆)alkyl;
R₇ is hydrogen, halogen, (C₁-C₆)alkoxy, fluoro(C₁-C₆)alkoxy, cyano, (C₁-C₆)alkyl, (C₂-C₆)alkenyl, (C₂-C₆)alkynyl; or fluoro(C₁-C₆)alkyl;
R₈ is hydrogen, halogen, or (C₁-C₆)alkyl, or, taken together with the carbon to which they are attached, R₈ and R₉ may form a 3- to 5-membered aliphatic carbocyclic ring;
R⁹ is hydrogen, halogen, or (C₁-C₆)alkyl, or, taken together with the carbon to which they are attached, R₈ and R⁹ may form a 3- to 5-membered aliphatic carbocyclic ring;
R₁₀ is hydrogen, halogen, (C₁-C₆)alkyl, (C₂-C₆)alkenyl, or (C₂-C₆)alkynyl, or, taken together along with the atoms to which they are attached,
wherein
(a) when R₂ is (C₁-C₆)alkyl, not all of R₄, R₅, R₆, and R₇ can be hydrogen;
(b) when R₁ is hydrogen, R₂ is methyl and R₅ is halogen, R₇ cannot be H or chloro; and
(c) when R₂ is (C₁-C₆)alkyl, and R⁵ is methoxy or carboxy, R⁷ cannot be H
wherein the drug is

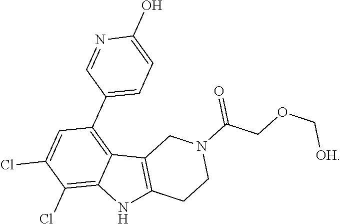 [SMILES: O=C(COCO)N1CCc2[nH]c3c(Cl)c(Cl)cc(-c4ccc(O)nc4)c3c2C1]

In some embodiments, the present invention provides a compound having the structure:

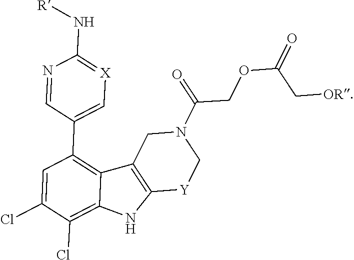

In some embodiments, R′ and R″ are each independently unsubstantiated or substituted amino acids.

In some embodiments, R′ and R″ are each independently unsubstantiated amino acids.

In some embodiments, R′ and R″ are unsubstantiated amino acids.

In some embodiments, R′ and R″ are alanine.

The present invention provides a pharmaceutical composition comprising the compound described in the present invention and a pharmaceutically acceptable carrier.

The present invention provides a method of sensitizing tumors to immune checkpoint blockade using the compound described in the present invention or the pharmaceutical composition described in the present invention.

In some embodiments of the method, the tumors have chromosomal instability.

The present invention provides a method for inhibiting an inflammatory response in a patient, wherein the method comprises administering to the patient in need thereof a therapeutically effective amount of the compound described in the present invention or the pharmaceutical composition described in the present invention.

The present invention provides a method for treating cancer metastasis in a patient, wherein the method comprises administering to the patient in need thereof a therapeutically effective amount of the compound described in the present invention or the pharmaceutical composition described in the present invention.

In some embodiments of the method, the cancer is lung cancer, breast cancer, prostate cancer, cervical cancer, pancreatic cancer, colon cancer, ovarian cancer, stomach cancer, esophagus cancer, skin cancer, heart cancer, liver cancer, bronchial cancer, testicular cancer, kidney cancer, bladder cancer, spleen, cancer, thymus cancer, thyroid cancer, brain cancer, or gall bladder cancer. In treating cancer or tumors as described herein, the following are other cGAS inhibitors that can be employed in the methods:
G140: A potent and selective inhibitor of human cGAS
G150: A highly selective human cGAS small-molecule
TD1006570 (TDI-6570): A prominent, blood-brain barrier-permeable, orally active inhibitor
RU.521: competitive inhibitor targeting the cGAS catalytic site;
CU-76/CU-32: Small-molecule cGAS antagonists that disrupt cGAS dimerization.
PF-06928215: A high-affinity inhibitor that mimics the interaction of the intermediate.
XL-3158/XL-3156: Potent, cross-species allosteric inhibitors that disrupt cGAS-DNA phase separation.
hydroxychloroquine and quinacrine
Vent-03 by Ventus Therapeutics
UM-200 (Barasa et al., J. Med. Chem. 2026, 69, 7, 8561-8582, hereby incorporated by reference)
IMSB301 (ImmuneSensor Therapeutics, Dallas, TX
cGAS inhibitors described in U.S. Ser. No. 10/329,258 B2, U.S. Pat. No. 12,433,897 B2, WO 2024/083773 A1 and WO 2025/172882 A1, hereby incorporated by reference.

In embodiments, the subject has not been diagnosed as having, and/or treated with a therapy for, an autoimmune condition.

The present invention provides a method of inhibiting inflammation, dsDNA-triggered interferon expression, or cGAS in a patient comprising administering the compound described in the present invention or the pharmaceutical composition described in the present invention.

The present invention provides a method of treating Aicardi Goutières Syndrome, Parkinson's disease, Alzheimer's disease, Systemic lupus erythematosus, or cancer metastasis in a patient comprising administering the compound described in the present invention or the pharmaceutical composition described in the present invention.

The present invention provides a medicament comprising the compound described in the present invention or the pharmaceutical composition described in the present invention and optionally a therapeutic agent for Aicardi Goutières Syndrome, Parkinson's disease, Alzheimer's disease, Systemic lupus erythematosus, or cancer metastasis.

The compounds of the present invention include all hydrates, solvates, and complexes of the compounds used by this invention. If a chiral center or another form of an isomeric center is present in a compound of the present invention, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present invention are in racemic form or as individual enantiomers. The enantiomers can be separated using known techniques, such as those described in Pure and Applied Chemistry 69, 1469-1474, (1997) IUPAC. In cases in which compounds have unsaturated carbon-carbon double bonds, both the cis (Z) and trans (E) isomers are within the scope of this invention.

The compounds of the subject invention may have spontaneous tautomeric forms. In cases wherein compounds may exist in tautomeric forms, such as keto-enol tautomers, each tautomeric form is contemplated as being included within this invention whether existing in equilibrium or predominantly in one form.

In the compound structures depicted herein, hydrogen atoms are not shown for carbon atoms having less than four bonds to non-hydrogen atoms. However, it is understood that enough hydrogen atoms exist on said carbon atoms to satisfy the octet rule.

This invention also provides isotopic variants of the compounds disclosed herein, including wherein the isotopic atom is ²H and/or wherein the isotopic atom ¹³C. Accordingly, in the compounds provided herein hydrogen can be enriched in the deuterium isotope. It is to be understood that the invention encompasses all such isotopic forms.

It is understood that the structures described in the embodiments of the methods hereinabove can be the same as the structures of the compounds described hereinabove.

It is understood that where a numerical range is recited herein, the present invention contemplates each integer between, and including, the upper and lower limits, unless otherwise stated.

Except where otherwise specified, if the structure of a compound of this invention includes an asymmetric carbon atom, it is understood that the compound occurs as a racemate, racemic mixture, and isolated single enantiomer. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The subject invention is also intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notation of a carbon in structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notation of a hydrogen in structures throughout this application, when used without further notation, are intended to represent all isotopes of hydrogen, such as ¹H, ²H, or ³H. Furthermore, any compounds containing ²H or ³H may specifically have the structure of any of the compounds disclosed herein.

Isotopically-labeled compounds can generally be prepared by conventional techniques known to those skilled in the art using appropriate isotopically-labeled reagents in place of the non-labeled reagents employed.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano, carbamoyl and aminocarbonyl and aminothiocarbonyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2 . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₂ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl, C₃-C₁₂ alkenyl, C₄-C₁₂ alkenyl and so on.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl, C₃-C₁₂ alkynyl, C₄-C₁₂ alkynyl and so on.

“Alkylene”, “alkenylene” and “alkynylene” shall mean, respectively, a divalent alkane, alkene and alkyne radical, respectively. It is understood that an alkylene, alkenylene, and alkynylene may be straight or branched. An alkylene, alkenylene, and alkynylene may be unsubstituted or substituted.

As used herein, “heteroalkyl” includes both branched and straight-chain saturated aliphatic hydrocarbon groups having the specified number of carbon atoms and at least 1 heteroatom within the chain or branch.

As used herein, “heterocycle” or “heterocyclyl” as used herein is intended to mean a 5- to 10-membered nonaromatic ring containing from 1 to 4 heteroatoms selected from the group consisting of O, N and S, and includes bicyclic groups. “Heterocyclyl” therefore includes, but is not limited to the following: imidazolyl, piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also encompassed by this definition.

As herein, “cycloalkyl” shall mean cyclic rings of alkanes of three to eight total carbon atoms, or any number within this range (i.e., cyclopropyl, cyclobutyl, cyclopentyl, cyclohexyl, cycloheptyl or cyclooctyl).

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to 10 atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include phenyl, p-toluenyl (4-methylphenyl), naphthyl, tetrahydro-naphthyl, indanyl, biphenyl, phenanthryl, anthryl or acenaphthyl. In cases where the aryl substituent is bicyclic and one ring is non-aromatic, it is understood that attachment is via the aromatic ring.

As used herein, the term “polycyclic” refers to unsaturated or partially unsaturated multiple fused ring structures, which may be unsubstituted or substituted.

The term “arylalkyl” refers to alkyl groups as described above wherein one or more bonds to hydrogen contained therein are replaced by a bond to an aryl group as described above. It is understood that an “arylalkyl” group is connected to a core molecule through a bond from the alkyl group and that the aryl group acts as a substituent on the alkyl group. Examples of arylalkyl moieties include, but are not limited to, benzyl (phenylmethyl), p-trifluoromethylbenzyl (4-trifluoromethylphenylmethyl), 1-phenylethyl, 2-phenylethyl, 3-phenylpropyl, 2-phenylpropyl and the like.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridizine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “alkylheteroaryl” refers to alkyl groups as described above wherein one or more bonds to hydrogen contained therein are replaced by a bond to an heteroaryl group as described above. It is understood that an “alkylheteroaryl” group is connected to a core molecule through a bond from the alkyl group and that the heteroaryl group acts as a substituent on the alkyl group. Examples of alkylheteroaryl moieties include, but are not limited to, —CH₂—(C₅H₄N), —CH₂—CH₂—(C₅H₄N) and the like.

The term “heterocycle” or “heterocyclyl” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The alkyl, alkenyl, alkynyl, aryl, heteroaryl and heterocyclyl substituents may be substituted or unsubstituted, unless specifically defined otherwise. In the compounds of the present invention, alkyl, alkenyl, alkynyl, aryl, heterocyclyl and heteroaryl groups can be further substituted by replacing one or more hydrogen atoms with alternative non-hydrogen groups. These include, but are not limited to, halo, hydroxy, mercapto, amino, carboxy, cyano and carbamoyl.

As used herein, the term “halogen” refers to F, Cl, Br, and I.

The terms “substitution”, “substituted” and “substituent” refer to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

In choosing the compounds of the present invention, one of ordinary skill in the art will recognize that the various substituents, i.e. R₁, R₂, etc. are to be chosen in conformity with well-known principles of chemical structure connectivity.

The various R groups attached to the aromatic rings of the compounds disclosed herein may be added to the rings by standard procedures, for example those set forth in Advanced Organic Chemistry: Part B: Reaction and Synthesis, Francis Carey and Richard Sundberg, (Springer) 5th ed. Edition. (2007), the content of which is hereby incorporated by reference.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. I. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

Some compounds of the invention may be synthesized from those found in USI 1414422B2 by using appropriate protecting reagents and conditions and standard peptide and ester coupling reagents and conditions. Some compounds of the invention may be synthesized from intermediates found in U.S. Ser. No. 11/414,422B2 by implementing commercially available reactants using standard reagents and conditions. Some compounds of the invention may be synthesized from similar intermediates and general reaction conditions found in U.S. Pat. No. 6,583,135B2 followed by the use of standard peptide and ester coupling reagents and conditions.

Another aspect of the invention comprises a compound used in the method of the present invention as a pharmaceutical composition.

In some embodiments, a pharmaceutical composition comprising the compound of the present invention and a pharmaceutically acceptable carrier.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department Of Health And Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat an infection or disease caused by a pathogen, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The compounds of the present invention may also form salts with basic amino acids such a lysine, arginine, etc. and with basic sugars such as N-methylglucamine, 2-amino-2-deoxyglucose, etc. and any other physiologically non-toxic basic substance.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier as are slow-release vehicles.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional antitumor agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or topically onto a site of disease or lesion, all using dosage forms well known to those of ordinary skill in the pharmaceutical arts.

The compounds used in the method of the present invention can be administered in admixture with suitable pharmaceutical diluents, extenders, excipients, or in carriers such as the novel programmable sustained-release multi-compartmental nanospheres (collectively referred to herein as a pharmaceutically acceptable carrier) suitably selected with respect to the intended form of administration and as consistent with conventional pharmaceutical practices. The unit will be in a form suitable for oral, nasal, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids such as lecithin, sphingomyelin, proteolipids, protein-encapsulated vesicles or from cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar-coated or film-coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, asuitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials such as solutol and/or ethanol to make them compatible with the type of injection or delivery system chosen.

The compounds and compositions of the present invention can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by topical administration, injection or other methods, to the afflicted area, such as a wound, including ulcers of the skin, all using dosage forms well known to those of ordinary skill in the pharmaceutical arts.

Specific examples of pharmaceutically acceptable carriers and excipients that may be used to formulate oral dosage forms of the present invention are described in U.S. Pat. No. 3,903,297 to Robert, issued Sep. 2, 1975. Techniques and compositions for making dosage forms useful in the present invention are described-in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

The active ingredient can be administered orally in solid dosage forms, such as capsules, tablets, powders, and chewing gum; or in liquid dosage forms, such as elixirs, syrups, and suspensions, including, but not limited to, mouthwash and toothpaste. It can also be administered parentally, in sterile liquid dosage forms.

Solid dosage forms, such as capsules and tablets, may be enteric-coated to prevent release of the active ingredient compounds before they reach the small intestine. Materials that may be used as enteric coatings include, but are not limited to, sugars, fatty acids, proteinaceous substances such as gelatin, waxes, shellac, cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), and methyl methacrylate-methacrylic acid copolymers.

The compounds and compositions of the invention can be coated onto stents for temporary or permanent implantation into the cardiovascular system of a subject.

Variations on those general synthetic methods will be readily apparent to those of ordinary skill in the art and are deemed to be within the scope of the present invention.

Each embodiment disclosed herein is contemplated as being applicable to each of the other disclosed embodiments. Thus, all combinations of the various elements described herein are within the scope of the invention.

Suppression of CIN or cGAS activity restores immunogenic potential of LKB1-deficient NSCLC. In experiments, relief of tonic STING activation via cGAMP could be removed by genetically deleting cGAS. Treatment of HCC44 with a commercially available human-selective cGAS inhibitor (G150) restored STING protein levels and enhanced pTBK1 and pIRF3 in response to cGAMP stimulation, thus, phenocopying genetic cGAS deletion experiments. Together, these results suggest that relief of tonic cGAS-STING activity through either suppression of CIN and genetic deletion or inhibition of cGAS restores productive immunogenic signaling in LKB1-deficient NSCLC.

Relief of CIN-induced signaling sensitizes NSCLC tumors to immune checkpoint blockade in vivo:. Following subcutaneous (s.c.) implantation of KP or KL models in immunocompetent mice, tumors were treated with anti-PD1 (aPD1) therapy or isotype control. Animals with KP tumors had a significant response and delay in tumor growth following aPD1 treatment, while KL did not Up-modulation of CIN rates through expression of dnMCAK in KP cells rendered these tumors completely resistant to aPD1 therapy, while downmodulation of CIN in KL, through the overexpression of MCAK, was sufficient to restore partial sensitivity to aPD1 therapy. Deletion of Cgas in the KL model reduced tumor growth, and substantially sensitized tumors to aPD1, leading to improved tumor control and animal survival. Together, these results demonstrate that CIN and its downstream sensor cGAS are sufficient and necessary, respectively, to determine response to immunotherapies.

Loss of Tumor cGAS Restores Sensitivity to Sting Agonists

To test if loss of tumor cell cGAS activity would sensitize otherwise resistant chromosomally unstable tumors to STING agonists, wild type or Cgas KO cells and treated tumors were transplanted biweekly with MSA-2. In four out of five models (KL, 4T1, CT26, and B16F10), treatment with MSA-2 significantly slowed the growth of Cgas KO tumors, extending the survival of treated animals. Importantly, these benefits were not observed in wildtype tumors, where MSA-2 treatment had a comparatively minimal effect Both cGAS proficient and deficient E0771.1mb tumors grew slightly slower in response to MSA-2, yet MSA-2 still prolonged survival of animals harboring Cgas KO tumors.

Pre-Treatment with cGAS Inhibitors Restores Tumor Sensitivity to STING Agonists

We next asked whether pharmacological inhibition of cGAS prior to—and during—treatment with STING agonists might represent a viable therapeutic strategy to overcome the resistance of chromosomally unstable tumors to STING activation. To that end, we made use of the cGAS inhibitor, RU.521, known for its stronger selectivity towards mouse cGAS as compared with human cGAS61 To confirm target inhibition, we incubated KL cells with RU.521 and measured expression of ISGs induction after stimulation with dsDNA. Unlike vehicle treated conditions, cells treated with RU.521 were unsuccessful in upregulating ISGs upon dsDNA stimulation. We next performed combination treatment of RU.521 and MSA-2 in all five previously described models and assessed for type I IFN induction. Indeed, pre-treatment with RU.521 significantly boosted ISG expression—except for Cxcl10—in response to MSA-2 exposure.

Given mounting evidence on reprograming of macrophages by STING agonists, macrophage polarization assays were carried out. Inhibition of cGAS in cancer cells led to reductions of Arginase 1 (an M2-like marker) in macrophages incubated with conditioned media from KL, 4T1, CT26 and B16F10 cells treated with MSA-2. Conversely, exposure of macrophages to conditioned media from MSA-2-treated Cgas KO cells led to a robust increase in iNOS (an M1-like marker) when compared to those exposed to media from wildtype MSA-2-treated cancer cells. Finally, KL, 4T1, and EO771.1mb were inoculated subcutaneously (KL) or orthotopically (4T1 and EO771.1mb) to evaluate the efficacy of RU.521 and MSA-2 combination treatment. Animals were pre-treated with RU.521 to ensure sufficient drug exposure by the time the first MSA-2 dose was administrated and continued RU.521 throughout the treatment course. Animals were treated with MSA-2 when tumors reached ~100 mm3. Expectedly, treatment with MSA-2 or RU.521 alone did not significantly hinder tumor growth in any of the models tested. On the other hand, combination treatment with MSA-2 and RU.521 led to a significant growth delay in KL, 4T1, and EO771.1mb tumors, thereby highlighting overcoming STING agonist resistance through cGAS inhibition.

1. A compound having the following structure: 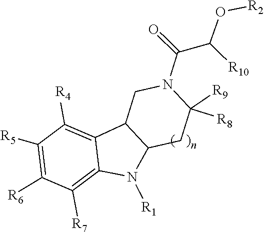
wherein
n is 1 or 2;
R₁ is hydrogen, (C₁-C₆)alkyl, (C₂-C₆)alkenyl, (C₂-C₆)alkynyl, CH₂CH₂OR₃, or fluoro (C₁-C₆)alkyl;
R₃ is hydrogen, halogen, (C₁-C₆)alkyl, (C₂-C₆)alkenyl, or (C₂-C₆)alkynyl;
R₅ is hydrogen, halogen, (C₁-C₆)alkyl, (C₂-C₆)alkenyl, (C₂-C₆)alkynyl, (C₁-C₆)alkoxy, fluoro(C₁-C₆)alkyl, fluoro(C₁-C₆)alkoxy, or cyano;
R₆ is hydrogen, halogen, cyano, (C₁-C₆)alkyl, (C₂-C₆)alkenyl, (C₂-C₆)alkynyl, or fluoro (C₁-C₆)alkyl;
R₇ is hydrogen, halogen, (C₁-C₆)alkoxy, fluoro(C₁-C₆)alkoxy, cyano, (C₁-C₆)alkyl, (C₂-C₆)alkenyl, (C₂-C₆)alkynyl, or fluoro(C₁-C₆)alkyl;
R₈ and R₉ are hydrogen, halogen, or (C₁-C₆)alkyl;
R₁₀ is hydrogen, halogen, (C₁-C₆)alkyl, (C₂-C₆)alkenyl, or (C₂-C₆)alkynyl;
wherein when n is 1:
R₂ is H, or a substituted or unsubstituted amino acid ester or α-hydroxy ester;
R₄ is a cyclic group substituted with one or more amines, or substituted with amino acids via the α-N
wherein
(i) when R₂ is H, R₄ is a cyclic group substituted with one or more amino acids via the α-N; and
(ii) when R₂ is a substituted or unsubstituted amino acid ester or α-hydroxy ester, R₄ is a cyclic group substituted with one or more amines, or substituted with amino acids via the α-N;
wherein when n is 2:
R₂ is a substituted or unsubstituted amino acid ester or α-hydroxy ester, hydrogen, (C₁-C₆)alkyl, (C₂-C₆)alkenyl, (C₂-C₆)alkynyl, or CH₂CH₂OR₁₁, or a 4- to 6-membered aliphatic ring containing an O;
R₁₁ is hydrogen, (C₁-C₆)alkyl, (C₂-C₆)alkenyl, or (C₂-C₆)alkynyl;
R₄ is hydrogen, halogen, (C₁-C₆)alkoxy, cyano, amino, an optionally substituted (C₁-C₆)alkyl, optionally substituted mono- or bicyclic heterocycle, optionally substituted phenyl, optionally substituted benzyl, (C₁-C₆) acylamino, or (C₁-C₆)N-alkyl carboxamido,
wherein
the optionally substituted alkyl, mono- or bicylclic heterocyle, phenyl, or benzyl groups are optionally substituted with amino, (C₁-C₆)mono- or dialkylamino, (C₁-C₆)alkoxy, or heterocyclic groups
or
a pharmaceutically acceptable salt or a pharmaceutically acceptable salt hydrate or deuterated analog thereof.

2. The compound or pharmaceutically acceptable salt or pharmaceutically acceptable salt hydrate or deuterated analog thereof of claim 1, wherein
(a) when R₂ is (C₁-C₆)alkyl, up to three of R₄, R₅, R₆, and R₇ can be hydrogen;
(b) when R₁ is hydrogen, R₂ is methyl, R₅ is halogen, and R₇ is a non-chloro halogen (C₁-C₆)alkoxy, fluoro(C₁-C₆)alkoxy, cyano, (C₁-C₆)alkyl, (C₂-C₆)alkenyl, (C₂-C₆)alkynyl, or fluoro(C₁-C₆)alkyl; and
(c) when R₂ is (C₁-C₆)alkyl, R⁵ is methoxy or carboxy, and R⁷ is halogen, (C₁-C₆)alkoxy, fluoro(C₁-C₆)alkoxy, cyano, (C₁-C₆)alkyl, (C₂-C₆)alkenyl, (C₂-C₆)alkynyl, or fluoro(C₁-C₆)alkyl.

3. The compound or pharmaceutically acceptable salt or pharmaceutically acceptable salt hydrate or deuterated analog thereof of claim 1, wherein R₁ is hydrogen or (C₁-C₆)alkyl, preferably R₁ is hydrogen.

4. The compound or pharmaceutically acceptable salt or pharmaceutically acceptable salt hydrate or deuterated analog thereof of claim 1, wherein when n is 1, R₂ is an unsubstituted or α-substituted amino acid ester or α-hydroxy ester, preferably the amino acid or α-hydroxy ester is substituted with amino, (C₁-C₆)alkylamino, (C₁-C₆)alkoxy, hydroxy, halogen, carboxyamido, methylenedioxy, ethylenedioxy, (C₁-C₆)acylamino, fluoro(C₁-C₆)acylamino, and hydroxy (C₁-C₆)alkylaminosulfonyl.

5. The compound or pharmaceutically acceptable salt or pharmaceutically acceptable salt hydrate or deuterated analog thereof of claim 1, wherein when n is 1, R₂ is an amino acid ester or α-hydroxy ester optionally unsubstituted or substituted on the α-heteroatom with an amino acid residue, α-hydroxy ester, α-(C₁-C₆)alkoxy ester (C₁-C₆)alkyl, (C₂-C₆)alkenyl, (C₂-C₆)alkynyl, amino(C₁-C₆)alkyl, hydroxy(C₁-C₆)alkyl, halo(C₁-C₆)alkyl, phenyl, benzyl, preferably R₂ is alanine.

6. The compound or pharmaceutically acceptable salt or pharmaceutically acceptable salt hydrate or deuterated analog thereof of claim 5, wherein R₂ is

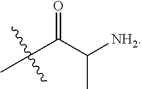 [SMILES: *C(=O)C(C)N]

7. The compound of or pharmaceutically acceptable salt or pharmaceutically acceptable salt hydrate or deuterated analog thereof of claim 5, wherein when n is 1, R₄ is a substituted mono- or bicyclic heterocycle or substituted phenyl.

8. The compound or pharmaceutically acceptable salt or pharmaceutically acceptable salt hydrate or deuterated analog thereof of claim 7, wherein the substituted mono- or bicyclic heterocycle or substituted phenyl is substituted with one or more amino acids via the α-N.

9. The compound or pharmaceutically acceptable salt or pharmaceutically acceptable salt hydrate or deuterated analog thereof of claim 8, wherein R₄ is a mono-substituted heteroaromatic monocycle substituted with an amino acid via the α-N.

10. The compound or pharmaceutically acceptable salt or pharmaceutically acceptable salt hydrate or deuterated analog thereof of claim 8, wherein the amino acid is alanine.

11. The compound or pharmaceutically acceptable salt or pharmaceutically acceptable salt hydrate or deuterated analog thereof of claim 10, wherein R₄ has the following structure: 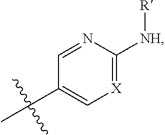
wherein X is C or N, preferably X is C;
wherein R′ is the amino acid.

12. The compound or pharmaceutically acceptable salt or pharmaceutically acceptable salt hydrate or deuterated analog thereof of claim 1, wherein, when n is 2, R₂ is an unsubstituted or α-substituted amino acid ester, hydrogen, (C₁-C₆)alkyl, (C₂-C₆)alkenyl, or (C₂-C₆)alkynyl, preferably R₂ is a substituted or unsubstituted amino acid, hydrogen, or (C₁-C₆)alkyl, more preferably R₂ is unsubstituted amino acid, more preferably R₂ is alanine.

13. The compound or pharmaceutically acceptable salt or pharmaceutically acceptable salt hydrate or deuterated analog thereof of claim 12, wherein, R₂ is

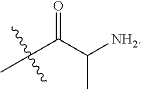 [SMILES: *C(=O)C(C)N]

14. The compound or pharmaceutically acceptable salt or pharmaceutically acceptable salt hydrate or deuterated analog thereof of claim 13, wherein R₃ is hydrogen, or (C₁-C₆)alkyl, preferably R₃ is hydrogen.

15. The compound or pharmaceutically acceptable salt or pharmaceutically acceptable salt hydrate or deuterated analog thereof of claim 14, wherein R₄ is an optionally substituted cyclic group.

16. The compound or pharmaceutically acceptable salt or pharmaceutically acceptable salt hydrate or deuterated analog thereof of claim 15, wherein the optionally substituted cyclic group may be substituted with one or more substituents independently chosen from: (C₁-C₆)alkyl, amino, cyano, (C₁-C₆)mono- or dialkylamino, (C₁-C₆)alkoxy, oxo, fluoro (C₁-C₆)alkyl, halogen, hydroxy, hydroxy(C₁-C₆)alkyl, (C₁-C₆)N-alkylcarboxamido, methylenedioxy, ethylenedioxy, (C₁-C₆)acylamino, fluoro(C₁-C₆)acylamino, an amino acid via the α-N and hydroxy (C₁-C₆)alkylaminosulfonyl, preferably an amino acid via the α-N.

17. A pharmaceutical composition comprising the compound of claim 1 and a pharmaceutically acceptable carrier.

18. A method of sensitizing a tumor to immune checkpoint blockade therapy comprising contracting the tumor with the compound of claim 1 or a small molecule cGAS inhibitor described herein.

19. The method of claim 18, wherein tumor has chromosomal instability.

20. A method for inhibiting cancer metastasis in a patient, wherein the method comprises administering to the patient in need thereof a therapeutically effective amount of the compound of claim 1 or a small molecule cGAS inhibitor described herein.
